(19) United States
(12) Patent Application Publication
(10) Pub. No.: US 2008/0139998 A1
(43) Pub. Date: Jun. 12, 2008

(54) Breastpump Assemblies Having Silver-Containing Antimicrobial Compounds

(75) Inventor: Brian H. Silver, Cary, IL (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Medela Holding AG, Baar (CH)

(21) Appl. No.: 11/952,659

(22) Filed: Dec. 7, 2007

Related U.S. Application Data
(60) Provisional application No. 60/873,701, filed on Dec. 8, 2006.

(51) Int. Cl. A61M 1/06

(52) U.S. Cl. 604/74

(57) ABSTRACT

Breastpump assemblies having antimicrobial agents associated therewith are disclosed. Embodiments include breastpump assemblies associated with elemental nanosilver. Other embodiments include breastpump assemblies associated with ionic silver in an ionic exchange resin.

This application claims benefit of U.S. Provisional Application No. 60/873,701, filed Dec. 8, 2006.

### FIELD OF THE INVENTION

The invention generally relates to breastpumps, and more particularly relates to antimicrobial agents for use in conjunction with breastpump assemblies.

### BACKGROUND OF THE INVENTION

Breastmilk pumps generally include a breastshield (also known as a suction hood) that typically includes a funnel-shaped surface sized and shaped to fit over the breast; a pressure source connected to the breastshield for generating an intermittent pressure (e.g., vacuum) within the breastshield; and conduit structure for communicating milk from the breastshield to a container for the expressed milk, as well as for communicating pressure variations (such as intermittent vacuum) to the breastshield. An example of this type of pump is shown in U.S. Pat. No. 5,007,899.

Breastshields, containers for the expressed milk, and associated tubing and valves are generally variously constructed of plastic, vinyl and/or rubber. Such materials have the advantage of being lightweight, easy to clean, durable, and relatively inexpensive to manufacture. In addition, such materials are able to withstand repeated exposure to high temperature and pressure, such as is used in cleaning and sterilizing the components.

During use, many of the breastpump components come into direct contact with the expressed breastmilk, other fluids, and obviously, the mother's body. As is well known, breastmilk is rich in nutrients, and constitutes an effective food source for not only infants, but microbes (e.g. bacteria and fungi) as well. The presence of such contaminants on the breastpump assembly can therefore lead to contamination of the expressed milk, which can lead to more rapid spoiling of the breastmilk, and further spread of the contaminants. This risk is exacerbated if the microbe for instance causing the contamination is a pathogen, or if the infant ingesting the contaminated breastmilk happens to be immuno-compromised and thus more susceptible to infection. Consequently, it is ordinarily recommended that breastpump components be thoroughly cleaned before and after use.

In a hospital setting, cleaning can include autoclaving the components of the breastpump assembly that come into contact with breastmilk. Autoclaving subjects the components to extremely high pressure and heat, and is effective to eliminate microbes from the assembly. Thereafter, the components may be sealed within sterile packaging for storage until use by a nursing mother. In settings outside a hospital or clinic, however, autoclave devices are generally not available or practical, and thus users ordinarily clean the breastpump assembly components with soap and water, or better still, a dishwasher unit.

While the above-described cleaning steps work well, they are not without certain disadvantages. One problem is the propensity for microbes and bacteria not removed or killed to nonetheless grow on surfaces following cleanings with soap and water. Microbes, and bacteria and fungi in particular, may have the ability to reside on surfaces of plastic, vinyl, and rubber for long periods in a relatively dormant state without substantial nutrients, and may in fact proliferate in such conditions, albeit relatively slowly. They may at least partially colonize the surfaces of a breastpump assembly following cleaning of the breastpump components, and thus are present when expressed milk comes in contact with the surfaces. Furthermore, strong antiseptic solutions or antibiotics are contraindicated, since those agents could mix with the expressed breastmilk and later be ingested by an infant. There thus remains a need, for an improved breastpump that itself helps to inhibit or prevent the growth of microbes on its surfaces.

### SUMMARY OF THE INVENTION

It is an aspect of the invention to provide a breastpump assembly including surfaces that have incorporated thereon or therein effective forms and amounts of antimicrobial materials. In one preferred embodiment, the antimicrobial materials include silver or silver-based compositions.

The invention may include an effective amount and form of silver and/or silver-containing compounds disposed on or provided to one or more parts or surfaces of the breastpump assembly. The antimicrobial composition may be provided as an additional layer or additional composition to the breastpump part or parts.

The silver or silver-containing compounds on the breastpump surfaces in one form of the invention release ions of silver that permeates the surfaces and contact the mother's body to confer antimicrobial qualities to the surfaces. The silver or silver containing compounds are preferably present on the breastpump assembly surfaces in sufficient quantities as to provide sufficient ionic silver at the breastpump assembly surface as to prevent colonization of microbes thereon. This could be a discrete outboard layer with the antimicrobial silver exposed therein. The antimicrobial compounds could also be applied to the breastpump in a fluid or cream or a similar separate base layer.

Advantages of embodiments of the invention include providing antimicrobial agents on the breastpump assembly surfaces that may be relatively permanent, (at least insofar as the typical lifetime of the unit when incorporated into the equipment), particularly with respect to elemental silver at the breastpump assembly surface, and thus present before and after use. Ionic forms of silver mixed in with the structural material of the breastpump can be adapted to migrate to breastpump assembly surfaces after use in sufficient concentrations to inhibit or eradicate microbes between uses of the breastpump assembly.

Another object of the invention is to provide methods of applying silver to breastpump assemblies and/or equipment associated with the storage and dispensing of breast milk. In certain embodiments, elemental nanosilver particles are attached to the surfaces of the breastpump assemblies. In other embodiments, the nanosilver particles are partially embedded in the surfaces of the breastpump assemblies. In yet other embodiments, ionic silver is applied to the surfaces of the breastpump assemblies.

Yet another embodiment of the invention includes the incorporation of an effective amount of silver-containing antimicrobial material into a lanolin or lanolin-containing composition. The combined lanolin and antimicrobial silver compound may be applied topically to a user before, during and/or after breastpump activity. The combined compound may be applied to the breastpump, for example, before the pump is used.

Yet another embodiment of the invention is the incorporation of antimicrobial agents containing effective amounts of silver or silver compounds into devices which while related to breast pumps or breastpump related functions, are not always considered part of a breastpump. For example, some commercially available breast pumps include flexible tubing to connect a pump part to a breast shield part. The invention contemplates the inclusion of silver-containing antimicrobial agents in the tubing. It should be understood that there are other ancillary parts, devices, and elements which are contemplated by the invention, when provided with silver-containing antimicrobial agents.

In addition to the incorporation or association of silver-containing antimicrobial agents in breast pumps and related devices, the invention contemplates the incorporation of silver-containing antimicrobial agents into breastfeeding equipment or accessories such as, for example, breast milk storage and feeding devices, such as bottles, collars, caps, lids and feeding nipples. Freezer bags, cooler carriers, pump valves, separation membranes, breastshield inserts, are all contemplated by the invention by incorporation of silver-containing antimicrobial agents.

### BRIEF DESCRIPTION OF THE DRAWING

FIG. 1 illustrates a side view of a breastpump assembly according to one embodiment of the invention.

### DETAILED DESCRIPTION

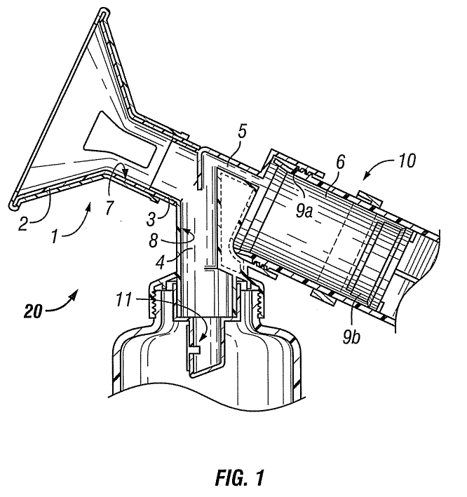

As shown in FIG. 1, an exemplary breastpump assembly 20 comprises a hood or breastshield 1. The breastshield has a funnel shape part 2 that during operation is placed over the breast of the mother. Another part 3 of the hood member is generally cylindrical in shape and communicates with a collecting or catch chamber 4, and with a vacuum line 6 via an extension 5. The vacuum line 6 leads to a pump 10, which can be manually or motor driven. A manual piston-type pump is shown, having a piston cylinder 9a and piston 9b. A catch container 12 (e.g. a bottle) may be attached to the second end via a threaded aperture, and so attached may collect expressed breastmilk.

As previously explained, surfaces of the breastpump assembly 20 have a number of surfaces, such as surfaces 7, 8, 11 on which breastmilk may be in contact or which contact the mother's body upon which microbes, bacteria, viruses, fungi and other living contaminants (referred to collectively simply as “microbes” hereafter for brevity, it being understood that all of these contaminants are in point), may colonize. In order to prevent or inhibit microbial proliferation on such surfaces, elemental silver or silver-containing compounds are embedded in or affixed to such surfaces. As explained in more detail below, elemental silver and certain silver-containing compounds exhibit antimicrobial properties, particularly when in the presence of moisture. The surface in particular point are those which contact the breast and breastmilk but the invention is applicable to all other desirable components, such as handles, tubing, etc.

Embodiments of the invention include compounds having silver, which may be applied to some or all of the components of the breastpump assembly to aid in the prevention of microbe colonization on the assembly components. Silver has long been known as possessing inherent antimicrobial properties, and as being safe for human contact and ingestion. Silver and silver-containing compounds are further known to be effective against a broad spectrum of microorganisms that cause, for example, disease, odor, and discoloration.

When present in aqueous solutions (i.e. in ionic form), silver has antimicrobial qualities due to the positively charged ionic form being highly toxic for microorganisms, but having relatively low toxicity for human cells. Specifically, silver ions have a high affinity for negatively charged side groups on biological molecules common in microbes, including sulfhydryl, carboxyl, phosphate, and other charged groups distributed throughout microbial cells. The binding reaction of silver to such side groups alters the molecular structure of the macromolecule, rendering it unusable to the microbial cell. Silver ions are further known to react with multiple sites within the microbial cell to inactivate critical physiological functions such as cell-wall synthesis, membrane transport, nucleic acid (such as RNA and DNA) synthesis and translation, protein folding and function, and electron transport, which is necessary for generating energy. Silver is thus a nonselective, broad spectrum antimicrobial agent also effective in the eradication of bacteria, fungi, and yeasts.

Surface-application methods can be employed to deposit either elemental silver or an ionic salt thereof to the surfaces of the breastpump assembly. Both forms are activated when placed in the presence of moisture. Ionic salts are active for relatively short periods of time, generally no more than a few days. Elemental particles of nanosilver, on the other hand, may persist in delivering antimicrobial forms of silver for as long as hundreds of days.

### Silver Zirconium Phosphate

In one embodiment, a zirconium phosphate-based ceramic ion-exchange resin containing silver is applied to the breastpump assembly components. An exemplary compound includes silver sodium hydrogen zirconium phosphate available from Milliken & Company under the name AlphaSan™. Formulations and application of ion-exchange resins containing silver are described in U.S. Pat. No. 7,118,761.

In a preferred embodiment, the silver sodium hydrogen zirconium phosphate is in an aqueous solution. The solution may be applied to any surface of the breastpump assembly that is desired to be free of microbes. In particular, surfaces 7, 8, 11 and any other surfaces that may contact expressed breastmilk may be coated with a layer of the solution containing the silver sodium hydrogen zirconium phosphate. So coated, the assembly 20 may be stored to maintain a relatively microbe-free state until the assembly 20 is used again. The silver antimicrobial solution could be provided in a kit, for example, where the mother can rinse, scrub or otherwise easily treat the surface(s).

### Nanosilver Particles

In another embodiment, nanosilver particles are coated onto or embedded in surfaces 7, 8, 11, for example, or any other desired surface of the breastpump assembly 20. Nanosilver particles are elemental silver particles measuring from 5 to 15 nm, and which function to facilitate the slow release of ionic silver into solution. When exposed to moisture, elemental silver oxidizes, resulting in the release of the ionic form. This chemical reaction occurs at the surface of the nanosilver particle. Because elemental silver oxidizes slowly, it is able to persist on the surface on which it is deposited device for longer periods of time than solutions containing silver compounds. Nanosilver particles have the advantage of having relatively large surface to volume ratios, which allow the nanosilver particles to release more ionic silver through oxidation than larger pieces of silver. By way of example, the surface area of one a gram of silver having a spherical configuration is 10.6 cm², compared to a gram of nanosilver particles having an average diameter of 10 nm and a surface area of 6×10⁵ cm². A number of well known methods may be used to adhere nanosilver particles to the desired surfaces of a breastpump assembly 20.

Nanosilver particles in either an aqueous or solvent-based solution may also be applied to surfaces of the breastpump assembly 20 to inhibit microbial proliferation. The chosen solution causes the outer layer of the nanosilver particles to oxidize upon exposure to air or fluids, forming a monolayer of silver oxide (Ag₂O) on the surface of each nanosilver particle. The silver oxide then dissolves in the fluid to produce the ionic (Ag⁺) form of silver, which is the form that is effective against microbes.

While endeavoring in the foregoing specification to draw attention to those features of the invention believed to be of particular importance it should be understood that the Applicants claim protection in respect of any patentable feature or combination of features hereinbefore referred to and/or shown in the drawings whether or not particular emphasis has been placed thereon. While the apparatus and method herein disclosed forms a preferred embodiment of this invention, this invention is not limited to that specific apparatus and method, and changes can be made therein without departing from the scope of this invention.

What is claimed is:

1. An improved breastshield for a breastpump, comprising:
a breastshield, said breastshield having a first inner surface adapted to receive a breast therein and direct breastmilk therethrough, and
an antimicrobial agent applied to at least a portion of said breastshield inner surface.

2. The breastshield of claim 1 wherein said antimicrobial agent is comprised of an antimicrobial form of silver.

3. The breastshield of claim 2 wherein said antimicrobial form of silver is exposed on said breastshield inner surface.

4. The breastshield of claim 2 wherein said antimicrobial form of silver is in part partially embedded in said breastshield, and said breastshield inner surface is of a material that permits the silver to migrate to said breastshield inner surface.

5. The breastshield of claim 1 wherein said antimicrobial agent is comprised of silver sodium hydrogen zirconium phosphate.

6. The breastshield of claim 1 wherein said antimicrobial agent is a nanosilver particulate material.

7. A method of conferring antimicrobial properties to a breastpump assembly, comprising:
providing one or more of a breastshield, other breastpump related components and breast feeding equipment, said breastshield and said other breastpump related components and breast feeding equipment having surfaces which come into contact with a mother's body or breastmilk in use, and
providing a contact active antimicrobial agent to at least portions of said one or more of a breastshield, said other breastpump related components and breast feeding equipment.

8. The method of claim 7 wherein said antimicrobial agent is comprised of an antimicrobial form of silver.

9. The method of claim 8, further comprising providing the antimicrobial form of silver in a layer on said surfaces.

10. The method of claim 9, wherein said providing of said antimicrobial form of silver is accomplished by applying a solution containing silver sodium hydrogen zirconium phosphate onto at least said breastshield inner surface.

11. The method of claim 8, wherein said one or more of a breastshield, said other breastpump related components and said breast feeding equipment is comprised of a material wherein said antimicrobial sliver is embedded for migration to an exposed surface thereof.

12. The method of claim 7, wherein said one or more of a breastshield, said other breastpump related components and breast feeding equipment includes one or more of bottles, collars, caps, lids, feeding nipples, freezer bags, cooler carriers, pump valves, separation membranes, and breastshield inserts.

13. The method of claim 7, wherein said agent includes lanolin.

14. A method of reducing microbial activity during breastpumping or breast feeding, comprising:
providing one or more of a breastshield and other breastpump or breast feeding related components with a contact active antimicrobial agent comprising silver.

15. A method of reducing microbial activity during breastpumping, comprising:
providing a lanolin composition with a contact active antimicrobial agent comprising silver to form a lanolin based antimicrobial agent; and
applying the lanolin based antimicrobial agent to one or more of a user, a breastshield and other breastpump related components.
